(19) United States
(12) Patent Application Publication
(10) Pub. No.: US 2017/0209223 A1
(43) Pub. Date: Jul. 27, 2017

(54) Instrument Annular Location Sensor

(71) Applicant: Analogic Corporation, Peabody, MA (US)

(72) Inventor: John P O'Connor, Andover, MA (US)

(21) Appl. No.: 15/328,966

(22) PCT Filed: Jul. 25, 2014
(86) PCT No.: PCT/US2014/048185

§ 371 Date: Jan. 25, 2017

(51) Int. Cl. A61B 34/20; A61B 10/02; A61B 18/14; A61B 8/08

(52) U.S. Cl. CPC A61B 34/20; A61B 8/0841; A61B 10/02; A61B 18/1492; A61B 8/483; A61B 2018/00577

(57) ABSTRACT

A system (100) includes an elongate instrument (102) having a long axis (104). The elongate instrument includes a first end portion (106; 202) along the long axis. The elongate instrument further includes a second end portion (108; 208) along the long axis, opposing the first end portion. The elongate instrument further includes an annular location determining device (118) disposed to surround a perimeter of a surface (122; 210) of a sub-region (124; 212) of the first end portion about the long axis. The annular location determining device generates a signal indicative of a three dimensional location of the annular location determining device, which indicates of a three dimensional location of the first end portion.

### TECHNICAL FIELD

The following generally relates to determining a location of an instrument in three dimensional space and more particularly to an instrument with an annular location sensor that generates information indicative of a location of the instrument in three dimensional space, and is described with particular application to ultrasound (US) imaging; however, the following is also amenable to other medical and/or non-medical imaging modalities.

### BACKGROUND

An ultrasound imaging apparatus has included a transducer array that transmits an ultrasound beam into an examination field of view. As the beam traverses structure (e.g., an object or subject, an instrument, etc.) in the field of view, sub-portions of the beam are attenuated, scattered, and/or reflected off the structure, with some of the reflections (echoes) traversing back towards the transducer array. The transducer array receives and processes the echoes, and generates one or more images of the subject or object and/or instrument. Ultrasound imaging has been used to guide medical procedures such as biopsies, ablations, laparoscopic, and/or other medical and/or non-medical procedures.

With medical procedures such as biopsies, ablations, laparoscopic, and/or other medical and/or non-medical procedures in which an instrument is inserted into a closed container where its location in the container is not readably discernable within the human, it is often desirable to monitor and/or track the location of the instrument in the container. One approach to monitoring and/or tracking the location of the instrument in the container has been to place one or more sensors on a side and/or handle of the instrument and employ a monitoring and/or tracking system the receives a signal from the sensor(s), processes the signal, and determines the location of the instrument in the container based on a result of the processing.

Unfortunately, such a sensor(s) is not well-suited for tracking the instrument when the instrument bends in a direction orthogonal to the sensor. As such, there is a need for an alternative approaches for monitoring and/or tracking the location of such an instrument in a container.

### SUMMARY

Aspects of the application address the above matters, and others.

In one aspect, a system includes an elongate instrument having a long axis. The elongate instrument includes a first end portion along the long axis. The elongate instrument further includes a second end portion, opposing the first end portion. The elongate instrument further includes an annular location determining device disposed to surround a perimeter of a surface of a sub-region of the first end portion about the long axis. The annular location determining device generates a signal indicative of a three dimensional location of the annular location determining device, which indicates of a three dimensional location of the first end portion.

In another aspect, a method includes receiving a signal from an annular location determining device surrounding a long axis of a first end portion of an elongate instrument. The annular location determining device is inside of a closed container and the signal provides information of a three dimensional location of the annular location determining device. The method further includes processing the signal and determining the three dimensional location of first end portion in the closed container. The method further includes determining if the first end portion is at a region of interest in the closed container based on the three dimensional location. The method further includes providing first feedback indicating the first end portion is at the region of interest in the closed container in response to determining the first end portion is at the region of interest, and second feedback indicating the first end portion is at the region of interest in the closed container in response to determining the first end portion is at the region of interest.

In another aspect, a system includes a medical instrument including a tip and an annular location determining device that generates a signal with information indicative of a 3D location of the tip inside of a subject. The system further includes an ultrasound imaging device that generates an image and processes the signal, and displays the image with a graphical representation of at least the tip of medical instrument superimposed thereover.

Those skilled in the art will recognize still other aspects of the present application upon reading and understanding the attached description.

### BRIEF DESCRIPTION OF THE DRAWINGS

The application is illustrated by way of example and not limited by the figures of the accompanying drawings, in which like references indicate similar elements and in which:

FIG. 1 schematically illustrates a system with an elongate instrument with an annular location determining device disposed about a long axis of the instrument;

FIG. 2 schematically illustrates an example in which the elongate instrument includes a needle and the annular location determining device surrounds an entirety of a perimeter of the needle;

FIG. 3 schematically illustrates the example in which the annular location determining device disposed proximate a tip of the instrument;

FIG. 4 schematically illustrates the example in which the annular location determining device disposed distal to a tip of the instrument;

FIG. 5 schematically illustrates the example in which the annular location determining device disposed proximate a mid-region of the instrument;

FIG. 6 schematically illustrates an example in which the annular location determining device does not surround the entirety of the perimeter of the needle;

FIG. 7 schematically illustrates an example in which the annular location determining device includes two sub-devices that surround the perimeter of the needle;

FIG. 8 schematically illustrates an example in which the annular location determining device includes a plurality of segments sequentially aligned around the perimeter of the needle;

FIG. 9 schematically illustrates an example in which the annular location determining device is part of a sheath that removably installs over the elongate instrument;

FIG. 10 schematically illustrates an example the elongate instrument in connection with an imaging apparatus;

FIG. 11 schematically illustrates a first example the imaging apparatus;

FIG. 12 schematically illustrates a second example the imaging apparatus; and

FIG. 23 illustrates an example method in accordance with the elongate instrument with the annular location determining device as described herein.

### DETAILED DESCRIPTION

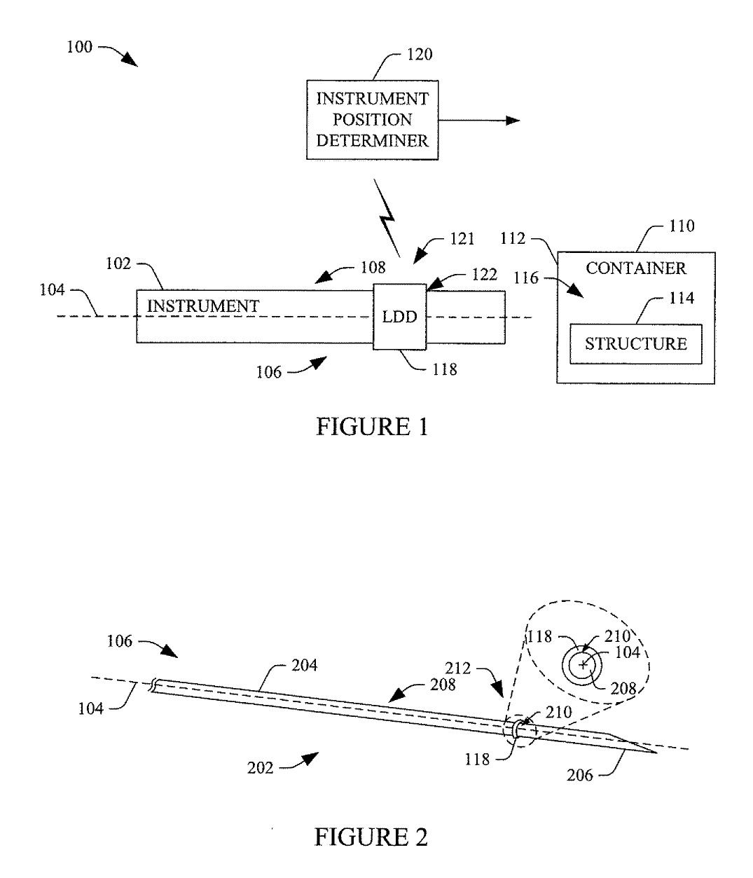

FIG. 1 schematically illustrates a system 100, which includes, at least, an elongate instrument 102 having a long axis 104 and including a first end portion 106 and a second end portion 108, which are located at opposing ends along the long axis 104. The instrument 102 is shown in connection with a container 110, which has a surface 112 and internal structure 114, and is closed in that an inside 116 of the container 110 is not readily visible from outside of the container 110 to the human eye.

The instrument 102, generally, can be any instrument in which one of the end portions 106 or 108 is configured for insertion into the container 110, it is desired to track and/or monitor the location and/or movement of the instrument 102 in the container 110, and the instrument 102 is not visible to the human eye when the instrument 102 is inside of the container 110. Examples of such an instrument for a medical application include, but are not limited to, a biopsy needle, an ablation catheter, and a laparoscopic probe.

For explanatory purposes, FIG. 2 shows an example in which the first end portion 106 includes an elongate needle 202 with a shaft 204 and a tip 206. The second end portion 108 (not shown) may include a handle, an instrument actuation mechanism, etc. For sake of brevity, other example instruments are not shown, but one of ordinary skill in the art, based at least in view of the description herein, would understand that the instrument can be another medical instrument such as an ablation catheter, a laparoscopic probe, etc., and/or a non-medical instrument.

Returning to FIG. 1, the instrument 102 includes a location determining device (“LDD”) 118 such as a sensor, a detector, and/or the like. The illustrated device 118 has an annular or closed-ring shape and completely surrounds a perimeter 122 of a sub-portion of the first end portion 106 about the long axis 104 of the instrument 102. The illustrated device 118 completely surrounds a middle region 121 of the end portion 106. In a variation, the device 118 is an open ring that surrounds less than the entire perimeter, e.g., from 50% (or 180 degrees) to less than 100% (or 360 degrees), such as 99.9%.

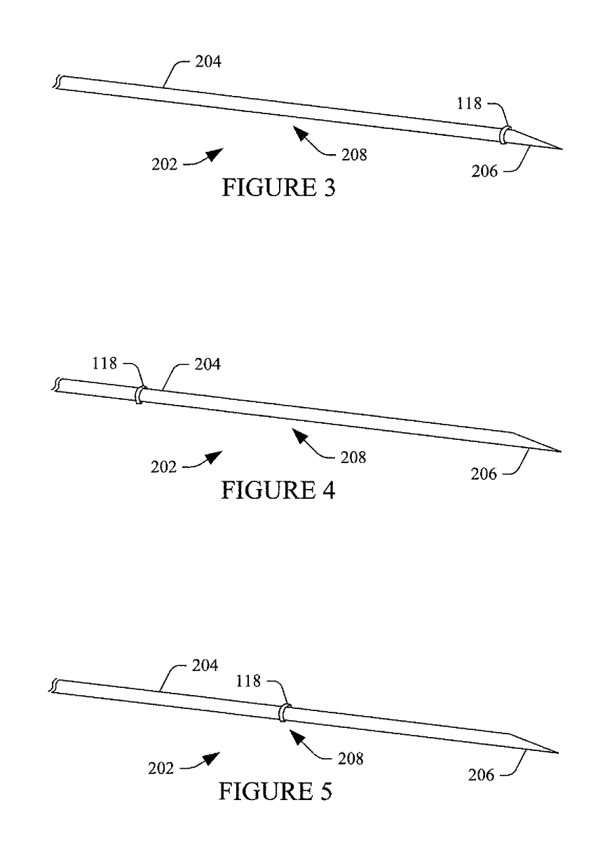

An example is shown in connection with FIG. 2. In FIG. 2, the device 118 is disposed proximate or adjacent to a surface 210 of a sub-portion 212 of the shaft 204 closer to the tip 206 than the second end portion 108. In other embodiments, the device 118 is disposed even closer to the tip 206 (e.g., at the tip 206), closer to the second end portion 108 than the tip 206, at a mid-region 208 of the first end portion 106, e.g., respectively as shown in FIGS. 3, 4 and 5.

Other locations for the device 118 are also contemplated herein. In FIGS. 2, 3, 4 and 5, the location determining device 118 is shown raised above the surfaces 122 and 210. In a variation, the device 118 can be even with the surfaces 122 and 210 and/or recessed within the surfaces 122 and 210. In addition, the instrument 102 may have more than one location determining device 118. It is also to be appreciated that the geometry and relative size of the device 118 is for explanatory purposes and is not limiting.

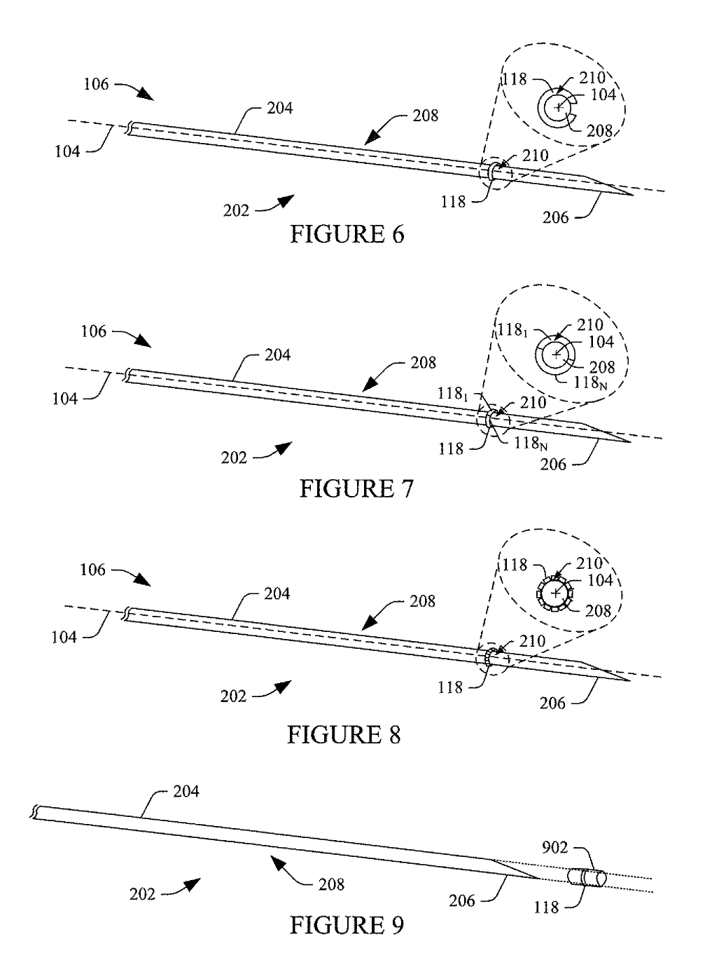

FIG. 6 shows an embodiment in which the device 118 does not completely surround (or only partially surrounds) the surface 210. FIG. 7 shows an embodiment in which the device 118 includes multiple sub-devices 118₁, . . . , 118_{N}, where N is a positive integer greater than two. In this example, N=2, and each sub-device is hemispherical in shape. In FIG. 8, the device 118 includes a plurality of linear segments 118₁, . . . , 118_{M}, where M is a positive integer greater than two, sequentially arranged about the surface 210 to forma ring.

Returning to FIG. 1, the device 118 can include an electric/electronic, a magnetic, an optical sensor and/or detector, a combination thereof, and/or other sensor and/or detector. Such devices may route signals therefrom through a physical communication path from the device 118, through the instrument 102, and to another device. Alternatively, the device 118 and/or the instrument 102 may include a wireless communication interface through which the signals are routed off the instrument 102. In either case, the signal from the device 118 will include location information.

In the illustrated example, the device 118 is built into and is part of the instrument 102. In a variation, the device 118 can be a separate component that attaches to the instrument 102. For example, in one non-limiting instance, the device 118 is part of a sheath or the like that installs over the first end portion 106. In one instance, the sheath is disposable. In another instance, the sheath is washable, disinfectable, and/or sterilizable, and re-usable. FIG. 9 shows an example in which the device 118 is part of a sheath 902.

Returning to FIG. 1, the illustrated device 118 generates a signal that includes information indicative of a three dimensional location of the device 118 and hence the first end portion 106. This includes the location of the first end portion 106 in three dimensional space, even in the event of bending of the first end portion 106 as the first end portion 106 passes through the surface 112 of the container 110 and/or the structure 114 within the container 110.

An instrument position determiner 120 receives the signal from the device 118 and determines, from the signal, the location and/or orientation of the device 118 in three dimensional space and, hence, the first end portion 106 in three dimensional space. The instrument position determiner 120 generates an output signal indicative of the location and/or orientation the first end portion 106 in three dimensional space. The output signal can be conveyed to a tracking system, a monitoring system, a robotic system, an imaging system, and/or other system.

Examples of suitable position determiner systems are described in U.S. patent application Ser. No. 12/703,706, filed Feb. 10, 2010, and entitled “Ultrasound Systems Incorporating Position Sensors and Associated Method,”, and U.S. patent application Ser. No. 12/775,403, filed May 6, 2010, and entitled “Freehand Ultrasound Imaging Systems and Methods for Guiding Elongate Instruments,” both which are incorporated herein by reference in their entireties. Other approaches are also contemplated herein.

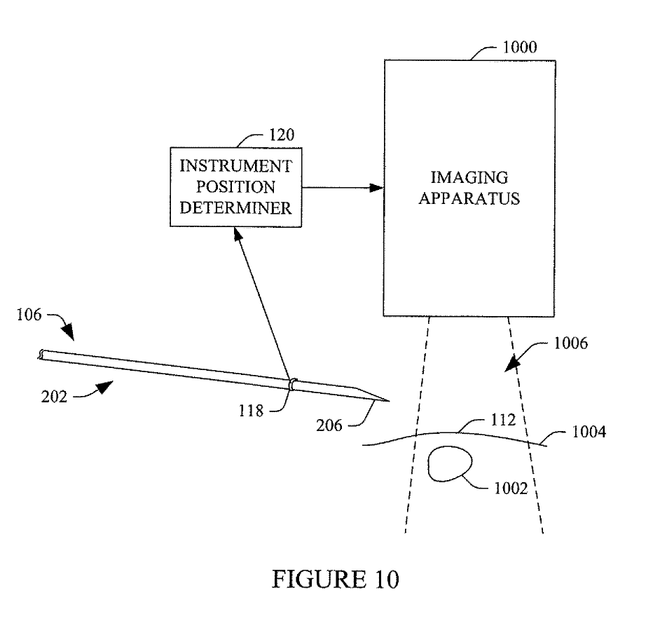

FIG. 10 shows an example with the instrument 102 described in connection with FIG. 2 in connection with an imaging apparatus 1000 for a biopsy procedure.

For this example, the tip 206 of the needle 202 is to be navigated or moved into an object or region of interest 1002 (e.g., tissue, fluid, etc.) of a patient 1004, which serves as the container 110 in this example. In this example, the needle 202, under guidance of images generated by the imaging apparatus 1000, is passed through the surface 112 and moved to tissue of interest 1002.

The imaging apparatus 1000 can be ultrasound (US), magnetic resonance (MR), computed tomography (CT), and/or other imaging apparatus, e.g., that generates imaging data which can be used to visually observe the needle 202 during an imaging-guided procedure such as a biopsy, a surgical, and/or other procedure. That is, an image generated will visually show the needle 202 and hence its location, if the needle 202 is within an imaging field of view 1006.

In one instance, the imaging apparatus 1000 receives the signal from the instrument position determiner 120 and displays the image with a graphical representation of the first end portion 106 superimposed thereover. The resulting image facilitates a user with navigating the first end portion 106 to the structure 114 in the container 110. In FIG. 10, this is equivalent to the image facilitating the tip 206 of the needle through the surface 112 of the patient 104 to the object of interest 1002. Examples of such representations are described in the '706 patent application and the '403 patent application. Other approaches are also contemplated herein.

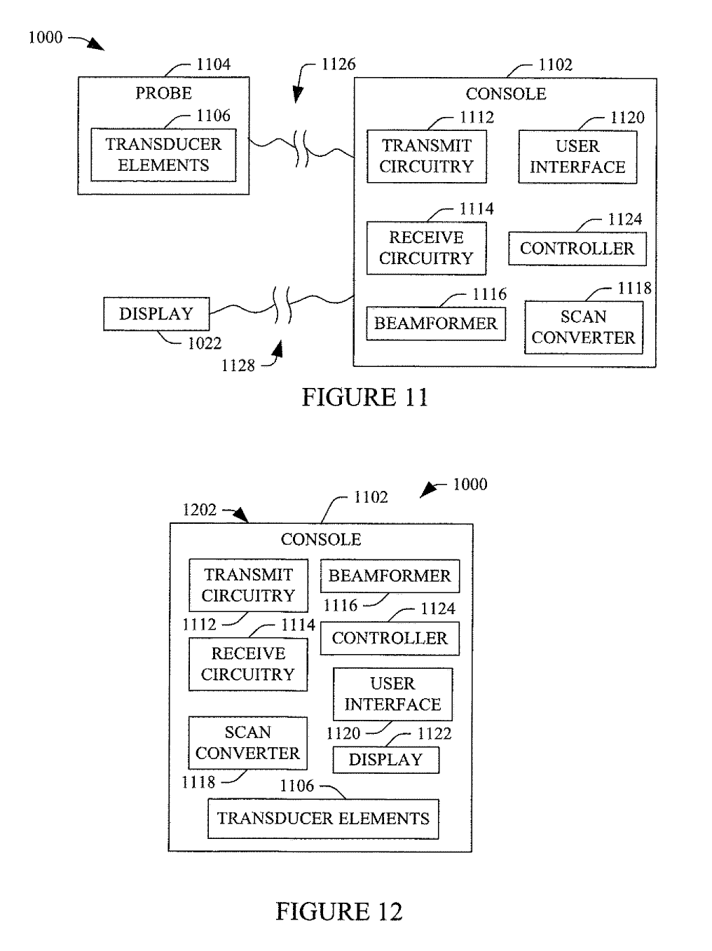

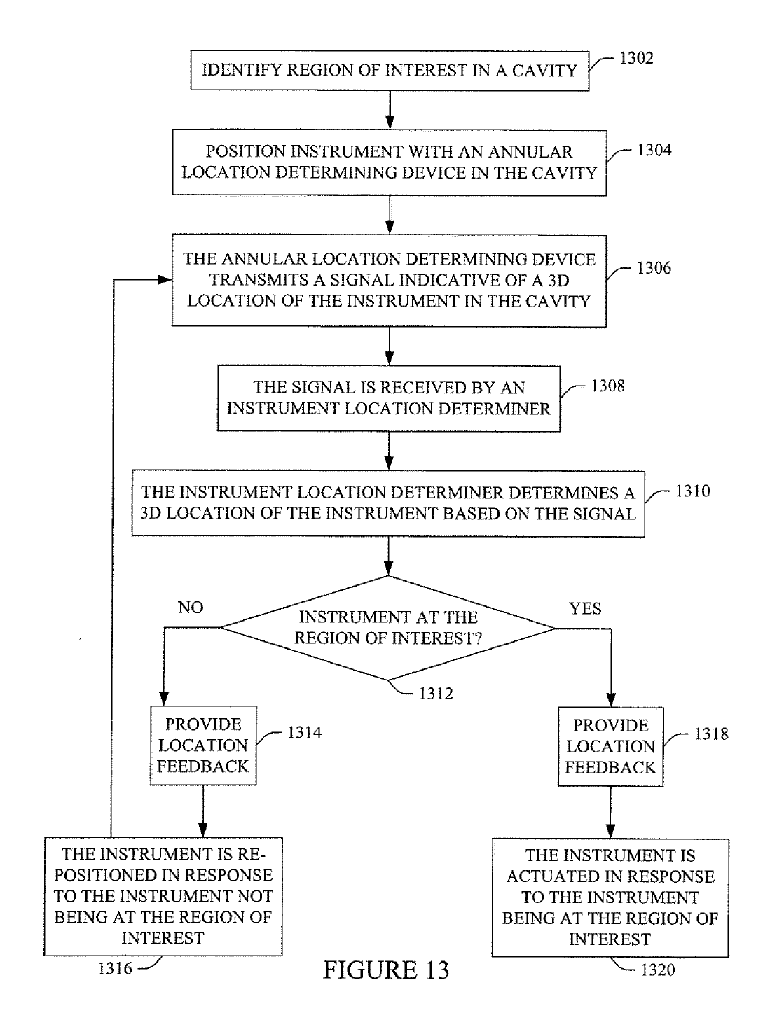

FIGS. 11 and 12 illustrate examples of the system 100 in which the imaging apparatus 1000 includes an US imaging device. The instrument 102 is not visible, not installed, etc. in FIGS. 11 and 12.

In FIG. 11, the imaging system 1000 includes a console 1102 and a separate US transducer probe 1004 that interfaces therewith. The ultrasound transducer probe 1104 includes a transducer array with a plurality of transducer elements 1106. The transducer array can be linear, curved, and/or otherwise shaped, fully populated, sparse and/or a combination thereof, etc. The transducer elements 1106 can be operated in 2D and/or 1D mode. The transducer elements 1106 transmit ultrasound signals and receive echo signals.

Transmit circuitry 1112 selectively actuates or excites one or more of the transducer elements 1106. More particularly, the transmit circuitry 1112 generates a set of pulses (or a pulsed signal) that are conveyed to the transducer elements 1106. The set of pulses actuates a set of the transducer elements 1106, causing the transducer elements 1106 to transmit ultrasound signals into an examination or scan field of view.

Receive circuitry 1114 receives a set of echoes (or echo signals) generated in response to the transmitted ultrasound signals. The echoes, generally, are a result of the interaction between the emitted ultrasound signals and the object (e.g., flowing blood cells, organ cells, etc.) in the scan field of view. The receive circuit 1114 may be configured for spatial compounding, filtering (e.g., FIR and/or IIR), and/or other echo processing.

A beamformer 1116 processes the received echoes. In B-mode, this includes applying time delays and weights to the echoes and summing the delayed and weighted echoes. A scan converter 1118 scan converts the data for display, e.g., by converting the beamformed data to the coordinate system of a display or display region used to visually present the resulting data.

A user interface (UI) 1120 include one or more input devices (e.g., a button, a knob, a slider, etc., touchscreen and/or physical mechanical device) and/or one or more output devices (e.g., a liquid crystal display, a light emitting diode, etc.), which allows for interaction with the system 100.

A display 1022 visually displays the US imaging data. A controller 1124 controls the various components of the imaging system 1000. For example, such control may include actuating or exciting individual or groups of transducer elements 1106 of the transducer array for B-mode, C-plane, etc.

The US probe 1104 and the display 1022 are physically separate electromechanical components with respect to the console 1102. The US probe 1104 and the display 1022 communicate with the console 1102 through communications paths 1126 and 1128. The communications paths 1126 and 1128 can be wired (e.g., a physical cable and connectors) and/or wireless.

FIG. 12 illustrates a variation of the US imaging system 1000. In this example, the console 1102 includes a single housing 1202. The single housing 1202 houses and physically supports the transducer elements 1106, the transmit circuitry 1112, the receive circuitry 1114, the beamformer 1116, the scan converter 1118 and the controller 1124, all of which are inside the single housing 1202, which is the physical mechanical casing of the console.

The user interface 1120 and/or the display 1122 can be part of the housing 1202. For example, the display 1122, in one instance, is a sub-portion of one of the sides of the housing 1202. The user interface 1120 may include physical mechanical controls at other locations on the housing 1202. The transducer elements 1106 are disposed in the housing 1202 behind an ultrasound window and emits ultrasound signals and receives echoes there through.

In FIG. 12, the US imaging system 1000 may be a hand-held ultrasound apparatus, which uses internally located power, e.g., from a power source such as a battery, a capacitor, etc. to power the components therein, and/or power from an external power source. An example of a hand-held device is described in U.S. Pat. No. 7,699,776 to Walker et al., entitled “Intuitive Ultrasonic Imaging System and Related Method Thereof,” and filed on Mar. 14, 2003, which is incorporated herein in its entirety by reference.

An example of hand-held ultrasound apparatus with an internal instrument guide is described in Ser. No. 13/017,344 to O'Connor, entitled “Ultrasound imaging apparatus,” and filed on Jan. 31, 2011, and an example with an external instrument guide is described in U.S. Pat. No. 8,226,562 to Pelissier, entitled “Hand-Held Ultrasound System Having Sterile Enclosure,” and filed on Aug. 7, 2008, both of which are incorporated herein in their entirety by reference.

The imaging systems in FIGS. 11 and 12 can be employed with an instrument guide, which holds the instrument 102. The instrument guide may be attached to the imaging systems 1000, separate therefrom, and/or internal to the imaging systems 1000. Furthermore, the instrument 102 can be utilized without the instrument guide, for example, free hand and/or by an electro-mechanical device such as a robotic arm.

FIG. 13 illustrates an example method for recording a location and an orientation of an interventional instrument at a point in time of a predetermined interventional event.

It is to be understood that the following acts are provided for explanatory purposes and are not limiting. As such, one or more of the acts may be omitted, one or more acts may be added, one or more acts may occur in a different order (including simultaneously with another act), etc.

At 1302, a region of interest is identified in a cavity.

At 1304, an instrument with an annular location determining device is positioned in a cavity.

At 1306, the annular location determining device transmits a signal indicative of a three dimensional location of the instrument in the cavity.

At 1308, the signal is received by an instrument location determiner.

At 1310, the instrument position determiner processes the signal, generating a location signal, which identifies the three dimensional location of the instrument in the cavity.

At 1312, it is determined whether the instrument is at the region of interest based on the identified the three dimensional location.

In response to determining the first end portion is not at the region of interest, at 1314, first feedback (e.g., visual, audible, etc.) indicating the first end portion is not at the region of interest in the closed container is provided, at 1316, the instrument is re-positioned in response to the instrument not being at the region of interest, and acts 1306 to 1312 are repeated.

In response to determining the first end portion is at the region of interest, at 1318, second feedback (e.g., visual, audible, etc.) indicating the first end portion is at the region of interest in the closed container is provided, and, at 1320, the instrument is actuated to perform a function.

The methods described herein may be implemented via one or more processors executing one or more computer readable instructions encoded or embodied on computer readable storage medium which causes the one or more processors to carry out the various acts and/or other functions and/or acts. Additionally or alternatively, the one or more processors can execute instructions carried by transitory medium such as a signal or carrier wave.

The application has been described with reference to various embodiments. Modifications and alterations will occur to others upon reading the application. It is intended that the invention be construed as including all such modifications and alterations, including insofar as they come within the scope of the appended claims and the equivalents thereof.

1. A system, comprising:
an elongate instrument having a long axis, the elongate instrument including:
a first end portion along the long axis;
a second end portion along the long axis, opposing the first end portion;
an annular location determining device disposed to surround a perimeter of a surface of a sub-region of the first end portion about the long axis,
wherein the annular location determining device generates a signal indicative of a three dimensional location of the annular location determining device, which indicates of a three dimensional location of the first end portion.

2. The system of claim 1, wherein the annular location determining device includes a closed ring that surrounds an entirety of the perimeter about the sub-region.

3. The system of claim 1, wherein the annular location determining device includes an open ring that does not surround an entirety of the sub-region.

4. The system of 1, wherein the annular location determining device includes at least two sub-portions that surround the perimeter of the sub-region.

5. The system of 1, wherein the annular location determining device includes a plurality of linear segments sequentially disposed about the surface of the sub-region.

6. The system of claim 1, wherein the annular location determining device is disposed at a first region of the first end portion.

7. The system of claim 6, wherein the annular location determining device includes a tip and an opposing region affixed to the second end portion, and the annular location determining device is at a first distance from the tip and a second distance from the second end portion, and the first distance is less than the second distance.

8. The system of claim 6, wherein the annular location determining device includes a tip and an opposing region affixed to the second end portion, and the annular location determining device is at a first distance from the tip and a second distance from the second end portion, and the first distance is greater than the second distance.

9. The system of claim 6, wherein the annular location determining device includes a tip and an opposing region affixed to the second end portion, and the annular location determining device is at a first distance from the tip and a second distance from the second end portion, and the first distance is equal to the second distance.

10. The system of 1, wherein the annular location determining device is part of sheath removably installable over the first end portion.

11. The system of 1, wherein the annular location determining device physically is part the first end portion of the elongate instrument.

12. The system of claim 1, further comprising:
an instrument position determiner that determines a position of the first end portion based on the signal indicative of the three dimensional location of the annular location determining device.

13. The system of any claim 1, wherein the instrument is one of a biopsy needle, an ablation catheter, or a laparoscopic device.

14. The system of claim 1, further comprising:
an imaging device that generates an image and displays the image with a graphical representation of the first end portion overlaid of the image at a position based on the signal.

15. A method, comprising:
receiving a signal from an annular location determining device surrounding a long axis of a first end portion of an elongate instrument, wherein the annular location determining device is inside of a closed container and the signal provides information of a three dimensional location of the annular location determining device;
processing the signal and determining the three dimensional location of first end portion in the closed container;
determining if the first end portion is at a region of interest in the closed container based on the three dimensional location; and
providing first feedback indicating the first end portion is at the region of interest in the closed container in response to determining the first end portion is at the region of interest, and second feedback indicating the first end portion is at the region of interest in the closed container in response to determining the first end portion is at the region of interest.

16. The method of claim 15, further comprising:
re-positioning the first end portion in response to the first feedback.

17. The method of any claim 15, further comprising:
actuating the elongate instrument to perform a first function in response to the second feedback.

18. The method of claim 17, wherein the first function is acquire a sample of tissue of interest at the region of interest.

19. The method of claim 17, wherein the first function is ablate tissue of interest at the region of interest.

20. A system, comprising:
a medical instrument including a tip and an annular location determining device that generates a signal with information indicative of a 3D location of the tip inside of a subject; and
an ultrasound imaging device that generates an image, processes the signal, and displays the image with a graphical representation of at least the tip of medical instrument superimposed thereover.
